(19) United States
(12) Patent Application Publication
(10) Pub. No.: US 2025/0134506 A1
(43) Pub. Date: May 1, 2025

(54) FISTULA BLOCKING STENT, STENT DELIVERING SYSTEM, AND METHOD OF DELIVERING FISTULA BLOCKING STENT

(71) Applicants: JIANGSU PROVINCE HOSPITAL, Nanjing (CN); MICRO-TECH (NANJING) CO., LTD., Nanjing (CN)

(72) Inventors: Yun Wang, Nanjing (CN); Jiwang Wang, Nanjing (CN); Guoxin Zhang, Nanjing (CN); Weifeng Zhang, Nanjing (CN); Lurong Li, Nanjing (CN); Jianyu Wei, Nanjing (CN); Zhenghua Shen, Nanjing (CN); Changqing Li, Nanjing (CN); Derong Leng, Nanjing (CN)

(21) Appl. No.: 18/757,709

(22) Filed: Jun. 28, 2024

(30) Foreign Application Priority Data
Oct. 31, 2023 (CN) 202311437850.3
Oct. 31, 2023 (CN) 202322948542.9

(51) Int. Cl. A61B 17/00; A61B 90/00

(52) U.S. Cl. CPC A61B 17/0057; A61B 17/00234; A61B 90/08; A61B 2017/00004; A61B 2017/00238; A61B 2017/00296; A61B 2017/00367; A61B 2017/00592; A61B 2017/00597; A61B 2017/00606; A61B 2017/00623; A61B 2017/00641; A61B 2017/00654; A61B 2017/00836; A61B 2017/00862; A61B 2090/0811

(57) ABSTRACT

A fistula blocking stent, a stent delivering system, and a method of delivering a fistula blocking stent are provided. The fistula blocking stent is configured to seal a fistula formed between a first lumen and a second lumen, which includes a first fixing stent in a shape of a hollow tube, the first fixing stent having a proximal end, a distal end, and a first tube extending axially between the proximal end and the distal end, the first fixing stent being provided in the first lumen; a second fixing stent provided in the fistula and the second lumen, the second fixing stent having a first end and a second end opposite to the first end, the first end being connected to the first fixing stent; and a barrier film provided on at least one of the first fixing stent and the second fixing stent and configured to seal the fistula.

### CROSS-REFERENCE TO RELATED APPLICATIONS

This application claims priority of Chinese Utility Model application No. 202322948542.9, filed on Oct. 31, 2023, and Chinese Patent Application No. 202311437850.3, filed on Oct. 31, 2023. The contents of the above identified applications are hereby incorporated herein in their entireties by reference.

### TECHNICAL FIELD

The present disclosure relates to the technical field of minimally invasive treatment, and in particular, to a fistula blocking stent, a stent delivering system, and a method of delivering a fistula blocking stent.

### BACKGROUND

Fistula is a disease that connects lumens of two adjacent organs due to congenital or acquired reasons, such as trauma, esophageal cancer, etc. For example, an esophagus-tracheal fistula is a fistula formed between an esophagus and a trachea to connect the esophagus and the trachea. The trachea is connected to a lung, and interiors of the two are sterile environments. When esophagus-tracheal fistula occurs, the substances in the esophagus, such as secretions, food, etc., will enter the trachea through the fistula, thus causing serious and difficult-to-treat pulmonary complications, such as aspiration pneumonia. In serious cases, patients cannot eat by mouth, which reduces the quality of life of patients.

The commonly used treatment method in the industry is to use a fistula blocking stent to seal the fistula. In prior art, the fistula blocking stent has poor sealing effect and causes complications.

### SUMMARY

Accordingly, it is necessary to provide a fistula blocking stent, a stent delivering system, and a method of delivering a fistula blocking stent.

A fistula blocking stent configured to block a fistula formed between a first lumen and a second lumen is provided. The fistula blocking stent includes a first fixing stent in a shape of a hollow tube, the first fixing stent having a proximal end, a distal end, and a tube body extending axially between the proximal end and the distal end, the first fixing stent being provided in the first lumen to support an inner wall of the first lumen; a second fixing stent provided in the fistula and the second lumen, the second fixing stent having a first end and a second end opposite to the first end, the first end being connected to the first fixing stent; and a barrier film provided on at least one of the first fixing stent and the second fixing stent and configured to block the fistula.

In one of the embodiments, the fistula blocking stent further includes a traction wire provided at the second end, and the second end is capable of being contracted by tightening the traction wire.

In one of the embodiments, when the traction wire is pulled, the second end is contracted to enable the second fixing stent to enter the second lumen through the fistula, when the traction wire is released, the second end is expanded to position the second fixing stent.

In one of the embodiments, the traction wire is circumferentially provided at the second end, when the second end is in a contracted state, a free section of the traction wire extends out of the second end is placed toward the distal end of the first fixing stent.

In one of the embodiments, the first fixing stent and the second fixing stent are connected by a suture.

In one of the embodiments, the barrier film includes a first covering film covering the first fixing stent, the second fixing stent is connected to the first fixing stent to form a first connection track, and the first covering film covers the first connection track.

In one of the embodiments, the barrier film includes a first barrier film connected to at least one of the first and second fixing stent, the second fixing stent is connected to the first fixing stent to form a first connection track, and the first barrier film covers the first connection track.

In one of the embodiments, the first barrier film is connected to the first fixing stent to form a second connection track, an area enclosed by the second connection track is greater than an area enclosed by the first connection track, and the first connection track is located within the second connection track.

In one of the embodiments, the first barrier film is an acid-resistant film.

In one of the embodiments, the barrier film further includes a second covering film covering the second fixing stent and a second barrier film provided in the second fixing stent, and the second barrier film is provided in a space surrounded by the second covering film to block the fistula.

In one of the embodiments, the first end is provided with a fistula blocking section, the second end is provided with a flange, a diameter of the flange is greater than a diameter of the fistula blocking section, and the second covering film covers the fistula blocking section.

In one of the embodiments, the first end is provided with a fistula blocking section, the second end is provided with a flange, the flange is in a shape of a hollow tube, and a central axis of the flange is parallel to a central axis of the first fixing stent.

In one of the embodiments, the first fixing stent is woven by metal wires, and the second fixing stent is woven by degradable wire or metal wires.

A stent delivering system is provided, which includes a delivering device including a first handle, a boosting tube connected to the first handle, a middle tube connected to the boosting tube, an inner tube connected to the middle tube, a pushing head connected to the inner tube, a second handle, an outer tube connected to the second handle, and a safety lock configured to lock or unlock a relative position between the second handle and the boosting tube, wherein the first handle is closer to an operator than the second handle, the booster tube extending through the second handle and being connected to the middle tube, the delivering device being a hollow structure, a diameter of the inner tube being less than a diameter of a distal end of the middle tube and a diameter of a proximal end of the pushing head, and the inner tube, the distal end of the middle tube, the proximal end of the pushing head, and the outer tube cooperatively form an accommodation space; and the foregoing fistula blocking stent provided in the accommodation space, wherein when the safety lock is released and the second handle is operated to retract the outer tube, the fistula blocking stent is released from the accommodation space.

In one of the embodiments, the fistula blocking stent further includes a traction wire circumferentially provided at the second end, when the second end is in a contracted state, a free section of the traction wire extends out of the second end and is placed toward the distal end of the first fixing stent, an outer surface of the outer tube is provided with a positioning mark corresponding to the free section.

A method of delivering a fistula blocking stent using the foregoing stent delivering system is provided. The method includes: contracting the fistula blocking stent into the accommodation space, and tightening locking the safety lock; inserting a cannula into the first lumen, enabling a first lumen endoscope to enter the first lumen through the cannula and pass a fistula, indwelling a guiding wire, removing the first lumen endoscope, and enabling the delivering device to enter the first lumen from the cannula through the guiding wire; inserting a second lumen endoscope to reach the fistula from the second lumen, unlocking the safety lock, operating the second handle to retract the outer tube to gradually release the fistula blocking stent, and when the traction wire is observed from the second lumen endoscope, pausing releasing of the fistula blocking stent, grabbing, by a forceps extending through a forceps channel of the second lumen endoscope and passing the fistula and entering the first lumen, the traction wire, and pulling the traction wire into the second lumen through the fistula; continuing to retract the outer tube to gradually release the fistula blocking stent, enabling the forceps to continuously apply a force to the traction wire toward the second lumen, so that the second fixing stent is gradually pulled into the second lumen through the fistula by the forceps during the gradual release of the fistula blocking stent, continuing to retract the outer tube until the proximal end of the first fixing stent is released from the outer tube; releasing, by the forceps, the traction wire, so that the second end of the second fixing stent is released to be expanded, wherein the second end is located in the second lumen and abuts against an inner wall of the second lumen, so as to prevent the second fixing stent from moving from the second lumen to the first lumen; and removing the delivering device and the guiding wire from the first lumen.

In one of the embodiments, when the second end is in a contracted state, a free section of the traction wire extends out of the second end is placed toward the distal end of the first fixing stent, an outer surface of the outer tube is provided with a positioning mark corresponding to the free section, wherein prior to operating the second handle to retract the outer tube to gradually release the fistula blocking stent, the method further includes: rotating the delivering device until the positioning mark is observed through the second lumen endoscope, and the positioning mark is orientated towards the fistula.

In one of the embodiments, the method further includes: injecting staining solution to a position of the second lumen adjacent to the fistula through the second lumen endoscope, enabling the first lumen endoscope to reach the fistula blocking stent from the first lumen, and determining whether the staining solution leaks into the first lumen from the second lumen through the first lumen endoscope.

A method of delivering a fistula blocking stent using the forgoing stent delivering system is provided. The method includes: contracting the fistula blocking stent into the accommodation space, and locking the safety lock; inserting a first lumen endoscope from the first lumen, enabling the first lumen endoscope to pass a fistula, indwelling a guiding wire, removing the first lumen endoscope, and enabling the delivering device to enter the first lumen through the guiding wire; inserting a cannula into the second lumen, inserting a second lumen endoscope into the second lumen from the cannula and enabling the second lumen endoscope to reach the fistula, unlocking the safety lock, operating the second handle to retract the outer tube to gradually release the fistula blocking stent, and when the traction wire is observed from the second lumen endoscope, pausing releasing of the fistula blocking stent, grabbing by a forceps extending through a forceps channel of the second lumen endoscope and passing the fistula and entering the first lumen, the traction wire, and pulling the traction wire into the second lumen through the fistula; continuing to retract the outer tube to gradually release the fistula blocking stent, enabling the forceps to continuously apply a force to the traction wire toward the second lumen, so that the second fixing stent is gradually pulled into the second lumen through the fistula by the forceps during the gradual release of the fistula blocking stent, continuing to retract the outer tube until the proximal end of the first fixing stent is released from the outer tube; releasing, by the forceps, the traction wire, so that the second end of the second fixing stent is released to be expanded, wherein the second end is located in the second lumen and abuts against an inner wall of the second lumen, so as to prevent the second fixing stent from moving from the second lumen to the first lumen; and removing the delivering device and the guiding wire from the first lumen.

In one of the embodiments, when the second end is in a contracted state, a free section of the traction wire extends out of the second end is placed toward the distal end of the first fixing stent, an outer surface of the outer tube is provided with a positioning mark corresponding to the free section, wherein prior to operating the second handle to retract the outer tube to gradually release the fistula blocking stent, the method further includes: rotating the delivering device until the positioning mark is observed through the second lumen endoscope, and the positioning mark is orientated towards the fistula.

In one of the embodiments, the method further includes: injecting the first lumen endoscope into the first lumen, injecting staining solution to a position of the first lumen adjacent to the fistula through the first lumen endoscope, and determining whether the staining solution leaks into the second lumen from the first lumen through the second lumen endoscope.

The fistula blocking stent is provided with the barrier film provided on at least one of the first fixing stent and the second fixing stent. The barrier film can separate the first fixing stent from the second fixing stent, so as to prevent liquid or food from flowing between the first lumen and the second lumen, thereby improving the blocking effect and reducing the possibility of causing complications.

### BRIEF DESCRIPTION OF THE DRAWINGS

To better describe and illustrate embodiments and/or examples of the present disclosure, reference may be made to one or more of the accompanying drawings. The additional details or examples used to describe the drawings should not be construed as limiting the scope of any of the disclosed disclosures, the embodiments and/or examples presently described, and the best modes currently understood of these disclosures.

FIG. 1 is a perspective view of a fistula blocking stent according to an embodiment.

FIG. 2 is a front view of the fistula blocking stent of FIG. 1.

FIG. 3 is a side view of the fistula blocking stent of FIG. 1.

FIG. 4 is a perspective sectional view of the fistula blocking stent of FIG. 1, showing that a second covering film completely covers a fistula blocking section.

FIG. 5 is a perspective sectional view of the fistula blocking stent of FIG. 1, showing that the second covering film does not completely cover the fistula blocking section.

FIG. 6 is a perspective view of a fistula blocking stent according to another embodiment.

FIG. 7 is a perspective view of a fistula blocking stent according to yet another embodiment.

FIG. 8 is a side view of the fistula blocking stent of FIG. 7.

FIG. 9 is a cross-sectional view of a delivering device according to an embodiment.

FIG. 10 is a perspective view of a stent delivering system according to an embodiment, showing that a distal end of the first fixing stent is released from the delivering device.

FIG. 11 is a perspective view of the stent delivering system according to an embodiment, showing that the distal end of the first fixing stent and a second fixing stent are released from the delivering device.

FIG. 12 is a perspective view of the stent delivering system according to an embodiment, showing that the fistula blocking stent is completely released from the delivering device.

FIG. 13 is an enlarged view of portion A in FIG. 10.

FIG. 14 is an enlarged view of portion B in FIG. 11.

FIG. 15 is an enlarged view of portion C in FIG. 12.

### DETAILED DESCRIPTION OF THE EMBODIMENTS

In order to make the above objects, features and advantages of the present disclosure clear and easier to understand, the specific embodiments of the present disclosure are described in detail below in combination with the accompanying drawings. Many specific details are set forth in the following description to facilitate a full understanding of the present disclosure. However, the present disclosure can be implemented in many ways different from those described herein, and those skilled in the art can make similar improvements without departing from the connotation of the present disclosure. Therefore, the present disclosure is not limited by the specific embodiments disclosed below.

In the following description, the terms “proximal end” refers to an end adjacent to an operator, and “distal end” refers to an end away from the operator.

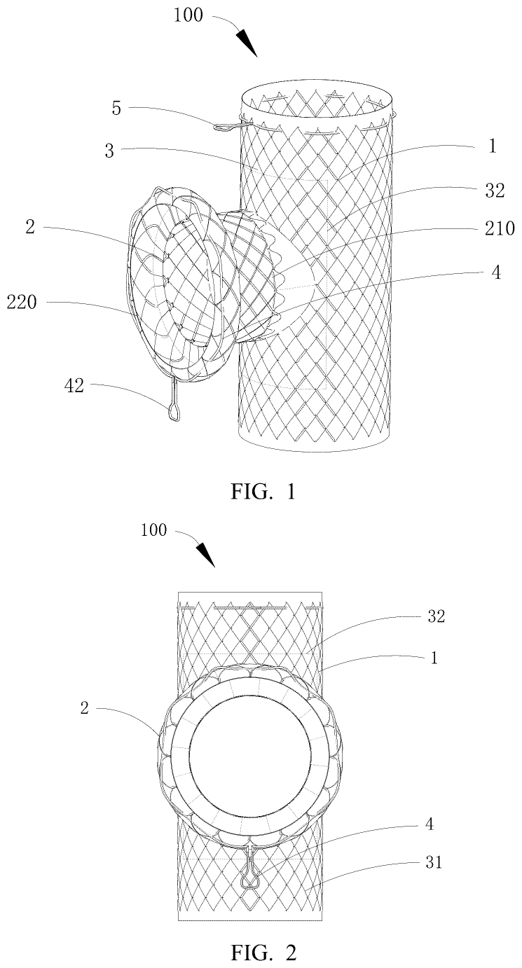

Referring to FIGS. 1 to 3, a fistula blocking stent 100 is provided according to an embodiment of the present disclosure, which is configured to block a fistula formed between a first lumen and a second lumen. The fistula blocking stent 100 includes a first fixing stent 1, a second fixing stent 2, and a barrier film 3 provided on at least one of the first fixing stent 1 and the second fixing stent 2. The barrier film 3 can separate the first fixing stent 1 from the second fixing stent 2, so as to prevent liquid or food from flowing between the first lumen and the second lumen, thereby improving the blocking effect and reducing the possibility of causing complications.

Specifically, both the first fixing stent 1 and the second fixing stent 2 are woven by threads, and the first fixing stent 1 and the second fixing stent 2 are connected to form a first connection track. The barrier film 3 at least covers the first connection track, thereby preventing tissue fluid or food from passing through the first fixing stent 1 and the second fixing stent 2 and flowing between the first lumen and the second lumen.

The first fixing stent 1 is provided in the first lumen to support an inner wall of the first lumen. The first fixing stent 1 is in a shape of a hollow tube, which has a proximal end, a distal end, and a tube body extending axially between the proximal end and the distal end. The second fixing stent 2 is provided in the fistula and the second lumen, the second fixing stent 2 has a first end 210 and a second end 220. A tube body of the second fixing stent 2 extends between the first end 210 and the second end 220, and the first end 210 is connected to the first fixing stent 1. After the fistula blocking stent 100 is placed in a patient, the fistula is sealed, thereby preventing substances such as liquid or food from flowing from one lumen to another lumen through the fistula.

The fistula blocking stent 100 configured to block the fistula generated between the esophagus and the trachea is described herein as an example, that is, the first lumen is one of an esophageal lumen and the tracheal lumen, the second lumen is the other of the esophageal lumen and the tracheal lumen. The tracheal lumen is in communication with a lung, interiors of the tracheal lumen and the lung are sterile environments, and other substances entering the lung may cause diseases such as pneumonia. The fistula blocking stent 100 is configured to block the fistula formed between the trachea and the esophagus to prevent substances such as secretions, food, etc. in the esophagus from entering the trachea lumen from the fistula and contaminating a respiratory tract, so as to control the occurrence of aspiration pneumonia.

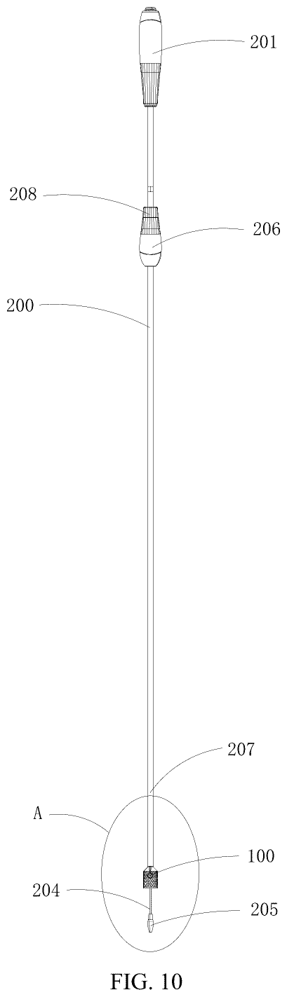

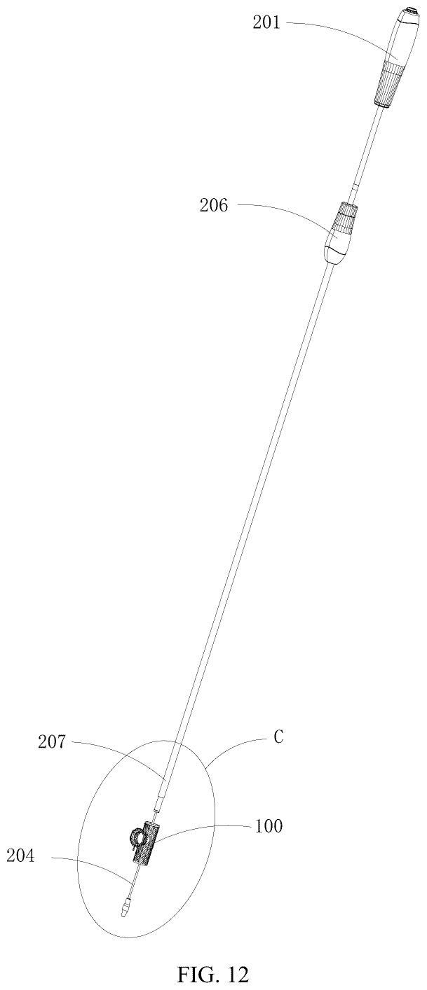

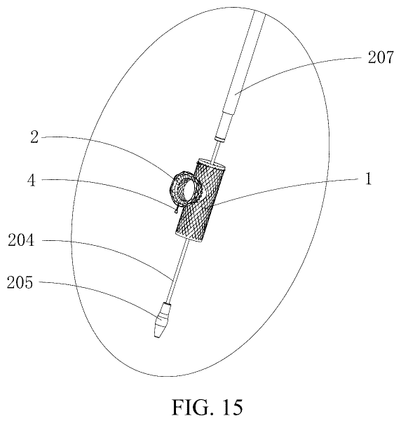

Referring to FIGS. 1 and 10 to 15, the fistula blocking stent 100 has an initial state, a contracted state, and a released state. Specifically, referring to FIG. 1, a state of the fistula blocking stent 100 not being contracted and not being placed in a delivering device 200 is defined as the initial state. Before the fistula blocking stent 100 is placed into the patient, the whole fistula blocking stent 100 is contracted and accommodated in a tubular structure of the delivering device 200, and the state of the fistula blocking stent 100 at this time is defined as the contracted state. Referring to FIGS. 10, 11, 13 and 14, when the fistula blocking stent 100 is delivered from the tracheal lumen or the esophageal lumen to a target area through the delivering device 200, the delivering device 200 is operated to slowly release the fistula blocking stent 100 from the delivering device 200. Under the elastic force and memory tension, a released part of the fistula blocking stent 100 gradually rebounds and recovers to the initial state, while an unreleased part of the fistula blocking stent 100 is still in the contracted state, and a state of the fistula blocking stent 100 at this time is defined as the released state. In an embodiment, the target area refers to an area where the fistula blocking stent 100 can begin to be released from the delivering device 200. Referring to FIGS. 12 and 15, the delivering device 200 is continuously operated until the fistula blocking stent 100 is completely released from the delivering device 200, and the completely released fistula blocking stent 100 recovers to the initial state. In the initial state, the fistula blocking stent 100 elastically abuts against inner walls of the trachea, the esophagus and the fistula, so that the fistula blocking stent 100 is fixed in the trachea and the esophagus, and the fistula is sealed.

In the following description, unless otherwise specified, the mentioned shape and size refer to a shape and a size of the fistula blocking stent 100 in the initial state.

Referring to FIGS. 1 and 2, a direction parallel to a central axis of the first fixing stent 1 is defined as a first direction, a direction parallel to a central axis of the second fixing stent 2 is defined as a second direction, the first direction and the second direction intersect with each other, and an angle between the first direction and the second direction ranges from 45° to 135°. In the first direction, the second fixing stent 2 is located in a middle of the first fixing stent 1. In an embodiment, the first direction and the second direction are perpendicular to each other. It should be noted that the parallel and perpendicular mentioned in the present disclosure are not absolutely parallel and absolutely perpendicular in the mathematical sense, but are substantially perpendicular and substantially parallel due to certain manufacturing errors and errors caused by deformation. Optionally, the error range is from −15° to 15°. The first fixing stent 1 is configured to be placed in one of the tracheal lumen and the esophageal lumen to fix a position of the fistula blocking stent 100 in the esophagus or the trachea, so as to prevent the fistula blocking stent 100 from moving relative to the trachea or the esophagus. The second fixing stent 2 is configured to extend through the fistula and is partially located in the other one of the tracheal lumen and the esophageal lumen to block the fistula and limit a position of the second fixing stent 2.

Before the fistula blocking stent 100 is delivered, the whole fistula blocking stent 100 is contracted in the tubular structure of the delivering device 200 in the contracted state, and the fistula blocking stent 100 is delivered from the first lumen to the target area by the delivering device 200. Then the delivering device 200 is operated to slowly deliver the fistula blocking stent 100, and the second fixing stent 2 is gradually delivered and placed in the second lumen after extending through the fistula under an action of external force. The delivering device 200 is continuously operated until the fistula blocking stent 100 is completely released from the delivering device 200. When the external force is removed, the second fixing stent 2 will expand under its own elastic force and tension. The expanded second fixing stent 2 is partially located in the fistula and abuts against the inner wall of the fistula to block the fistula, and the expanded second fixing stent 2 is partially located in the second lumen and abuts against the inner wall of the second lumen, so as to prevent the second fixing stent 2 from being separated from the second lumen. After the first fixing stent 1 is completely released, the first fixing stent 1 expands under its own elastic force and tension and recovers to the initial state. The first fixing stent 1 elastically abuts against the inner wall of the first lumen to prevent the fistula blocking stent 100 from moving relative to the first lumen, thereby positioning the fistula blocking stent 100.

In order to achieve a better fixation effect, when the fistula blocking stent 100 is delivered from the tracheal lumen, a diameter of the first fixing stent 1 is greater than a diameter of the tracheal lumen. In order to improve the comfort of the patient, the diameter of the first fixing stent 1 is slightly greater than the diameter of the tracheal lumen. In an embodiment, an outer diameter of the first fixing stent 1 is 8 mm to 24 mm and a length of the first fixing stent 1 is 25 mm to 45 mm. The outer diameter of the first fixing stent 1 may be 12 mm, 14 mm, 16 mm, 18 mm, 20 mm, 22 mm, or 24 mm, and a length of the first fixing stent 1 may be 40±5 mm, 30±5 mm, or 40±4 mm. When the first fixing stent 1 is configured to be fixed in the main trachea, the outer diameter of the first fixing stent 1 is 14 mm to 24 mm, and the length of the first fixing stent 1 is 40±5 mm or 30±5 mm. The outer diameter of the first fixing stent 1 may be 16 mm, 18 mm, 20 mm, 22 mm or 24 mm. When the first fixing stent 1 configured to be fixed in the bronchus, the outer diameter of the first fixing stent 1 is 8 mm to 16 mm, and the length of the first fixing stent 1 is 40±4 mm. Optionally, the outer diameter of the first fixing stent 1 may be 12 mm or 14 mm.

The first fixing stent 1 and the second fixing stent 2 are separated elements, that is, the first fixing stent 1 is connected to the second fixing stent 2 after the first fixing stent 1 and the second fixing stent 2 are manufactured respectively. In the embodiment, the first fixing stent 1 is sewn to the second fixing stent 2, and the first connection track is a first sewing track generated by sewing. Specifically, the first fixing stent 1 is connected to the second fixing stent 2 by a suture. In an embodiment, the suture is a PTFE (Polytetrafluoroethylene) wire.

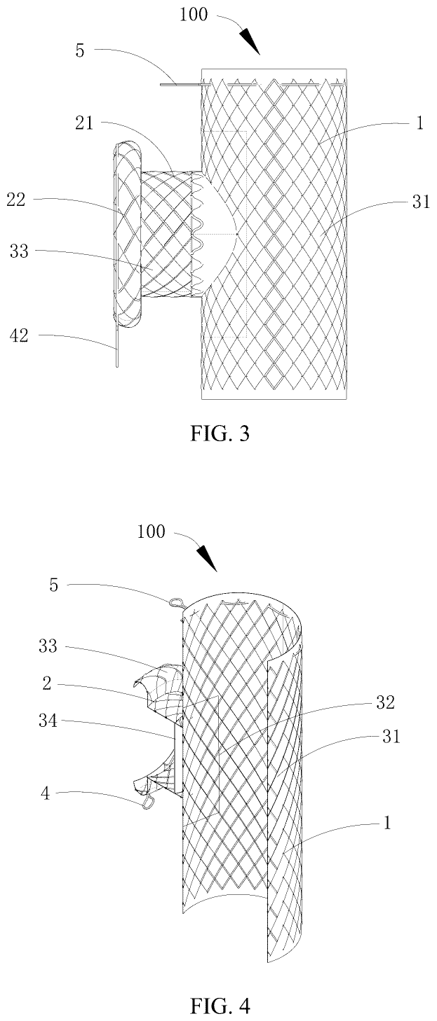

Referring to FIG. 3 and FIG. 4, the barrier film 3 includes a first covering film 31 covering the first fixing stent 1, and the first covering film 31 at least covers the first connection track. That is, the first covering film 31 completely covers the first end 210 of the second fixing stent 2, so as to prevent substances such as food, secretions, etc. from flowing between the two lumens through the fistula. The first covering film 31 is made of silicone, which can prevent substances such as food, secretions, etc. from entering an inner cavity the first fixing stent 1 from an inner cavity of the second fixing stent 2.

In an embodiment, the first covering film 31 and the first fixing stent 1 are integrally formed. Specifically, the manufactured first fixing stent 1 is immersed in a container containing coating solution, and after standing for a predetermined time, the first fixing stent 1 adhered with the coating solution is taken out and cured, so that the first covering film 31 and the first fixing stent 1 are integrally formed.

In the embodiment in which the first fixing stent 1 is sewn to the second fixing stent 2, the first connection track is provided with a plurality of first pinholes extends through the first covering film 31. Liquid in the esophageal lumen, such as tissue fluid, may enter the inner cavity of the first fixing stent 1 through the first pinholes, and then enter the tracheal lumen. The fluid will enter the lungs along the trachea, which will cause serious complications such as lung infection. In addition, the first covering film 31 is a silicone film, which has poor acid resistance. After the fistula blocking stent 100 is delivered into the patient for a period of time, the first covering film 31 may be corroded by the acid liquid in the esophagus, and when the first covering film 31 is damaged, the secretion, food, etc. in the esophagus will enter the trachea from a damaged part of the first covering film 31, which causes complications such as lung infection. Referring FIGS. 1 and 4, in order to prevent the liquid in the esophagus from entering the lungs from the first pinhole and/or the damaged part of the first covering film 31, the barrier film 3 further includes a first barrier film 32 connected to at least one of the first fixing stent 1 and the second fixing stent 2, and the first barrier film 32 at least covers the first connection track. In an embodiment, the first barrier film 32 is connected to the first fixing stent 1 and covers the first connection track.

The first barrier film 32 is connected to at least one of an inner side and an outer side of the first fixing stent 1, so that an accommodation cavity is formed between the first barrier film 32 and the first fixing stent 1. A track formed by connecting the first barrier film 32 and the first fixing stent 1 is defined as a second connection track. An area enclosed by the second connection track is greater than an area enclosed by the first connection track, and the second connection track completely surrounds the first connection track, so that the first connection track is located in the accommodation cavity. When the liquid flows into the first fixing stent 1 from the first pinhole and/or the damaged part of the first covering film 31, the liquid is stored in the accommodation cavity and does not flow into the trachea, so that the liquid can be effectively prevented from flowing into the lung from the first pinhole, thereby avoiding lung infection.

In the embodiment, the first barrier film 32 is connected to the first fixing stent 1 by a sewing process, and the second connection track is a second sewing track. During the sewing process, a plurality of second pinholes is formed on the first covering film 31 and the first barrier film 32, and the plurality of second pinholes are sealed with silicone to prevent fluid from entering the lung from the second pinholes.

In an embodiment, the first barrier film 32 is an acid-resistant film made of PTFE (Polytetrafluoroethylene), FEP (Fluorinated Ethylene Propylene) or ePTE (Polytetrafluoroethylene). In alternative embodiments, the barrier film 3 may be made of other acid-resistant materials.

Since the PTFE film has poor shrinkage property and poor elastic deformation property, if an area of the first barrier film 32 is too large, the delivery of the fistula blocking stent 100 will be hindered. Therefore, in the design, a size of the first barrier film 32 should be as small as possible while satisfying the function that the first barrier film 32 can prevent the liquid from entering the lung from the damaged part of the first pinhole or the first covering film 31.

Specifically, the first barrier film 32 does not completely cover the first fixing stent 1, but completely covers the first connection track, that is, the area of the first barrier film 32 is less than a surface area of the first fixing stent 1, but greater than an area surrounded by the first connection track.

In the embodiment, in the first direction, the size of the first barrier film 32 is less than a size of the first fixing stent 1 and greater than a size of the first connection track. In a circumferential direction of the first fixing stent 1, both ends of the first barrier film 32 are spaced from each other, that is, in the circumferential direction of the first fixing stent 1, the size of the first barrier film 32 is less than a circumference of the first fixing stent 1 and greater than the size of the first connection track, so that the first barrier film 32 can completely cover the first connection track. In the first direction and the circumferential direction of the first fixing stent 1, the first connection track is located in a middle of the first barrier film 32, so that the first barrier film 32 can completely cover the first connection track while minimizing the size of the first barrier film 32. In an embodiment, a certain sewing margin is reserved between an edge of the first barrier film 32 and the second connection track, which can ensure the sewing stability of the first barrier film 32.

In an alternative embodiment, in the circumferential direction of the first fixing stent 1, the first barrier film 32 may be continuous, that is, the first barrier film 32 is provided in a hollow cylindrical shape, so that the first barrier film 32 completely covers the surface of the first fixing stent 1 in the circumferential direction. Similarly, the size of the first barrier film 32 in the first direction is less than the size of the first fixing stent 1 and greater than the size of the first connection track. In this embodiment, while the first barrier film 32 is sewn to the first fixing stent 1 to form the second connection track, a third connection track is also formed. The third connection track does not intersect with the second connection track, and the third connection track completely surrounds the second connection track. The first barrier film 32 is connected to the first fixing stent 1 in multiple places, which can ensure the connection stability between the first barrier film 32 and the first fixing stent 1. A certain sewing margin is reserved between the edge of the first barrier film 32 and the third connection track, which can ensure the sewing stability of the first barrier film 32.

In alternative embodiments, the first barrier film 32 is provided between the first fixing stent 1 and the second fixing stent 2 and connected to the second fixing stent 2. Specifically, before the first fixing stent 1 is connected to the second fixing stent 2, the first barrier film 32 covers an opening of the first end 210 of the second fixing stent 2, and the first barrier film 32 is connected to the second fixing stent 2, then the second fixing stent 2 is connected to the first fixing stent 1, and the pinhole formed by sewing is sealed with silicone.

Referring to FIG. 3 and FIG. 4, in order to improve the blocking effect, the barrier film 3 further includes a second covering film 33 at least partially covering the second fixing stent 2. The second covering film 33 and the second fixing stent 2 are integrally formed. Specifically, the manufactured second fixing stent 2 is immersed in a container containing the coating solution, and after standing for a predetermined time, the second fixing stent 2 adhered with the coating solution is taken out and cured, so that the second covering film 33 and the second fixing stent 2 are integrally formed.

In order to further improve the blocking effect, the barrier film 3 further includes a second barrier film 34 provided in the second fixing stent 2, and the second barrier film 34 is provided in a space surrounded by the second covering film 33 to block the fistula.

Specifically, the first end 210 of the second fixing stent 2 is provided with a fistula blocking section 21. The second end 220 is provided with at least one flange 22, a diameter of the flange 22 is greater than a diameter of the fistula blocking section 21. The second film 33 covers the fistula blocking section 21 and the flange 22, and the second barrier film 34 is provided in at least one of the fistula blocking section 21 and the flange 22 to block the fistula. After the delivery of the fistula blocking stent 100 is completed, the fistula blocking section 21 is located in the fistula, the fistula blocking section 21, the second covering film 33, and the second barrier film 34 cooperatively support and seal the fistula. The flange 22 is located in the second lumen to limit the position of the fistula blocking stent 100, which can effectively prevent the flange 22 from moving from the second lumen to the first lumen, thereby effectively preventing the expansion fistula blocking stent 100 from moving.

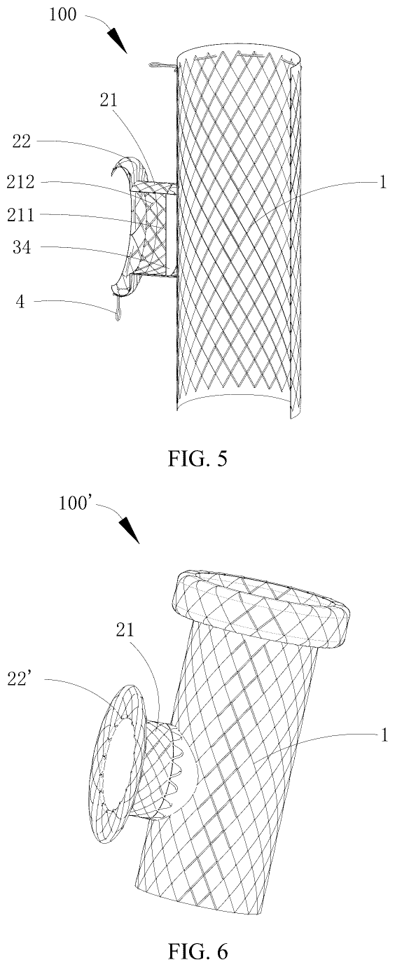

Referring to FIG. 5, in order to better promote the healing of the fistula, the second covering film 33 may not completely cover the fistula blocking section 21 of the second fixing stent 2, so that granulations at the fistula gradually passes through gaps between the woven threads of the fistula blocking section 21 and enters an interior the fistula blocking section 21 during the growth process, and finally the granulations grow together and squeeze part of the second fixing stent 2 to be separated from the first fixing stent 1, so as to facilitate the healing of the fistula. In this embodiment, the fistula blocking section 21 includes a hollow section 211 and a film covering section 212, and the hollow section 211 is closer to the first fixing stent 1 than the film covering section 212. The second film 33 is not provided on an inner side and an outer side of the hollow section 211, but is provided on the film covering section 212. The second barrier film 34 is provided in the space surrounded by the second covering film 33 of the covering film section 212 to block the fistula.

In an alternative embodiment, the barrier film 3 includes only a first covering film 31, and the plurality of first pinholes are sealed with silicone to ensure the blocking effect.

In an alternative embodiment, the barrier film 3 includes only the first barrier film 32 covering the first connection track.

In an alternative embodiment, the barrier film 3 includes only the second covering film 33, and the second barrier film 34 provided in the space enclosed by the second covering film 33 to block the fistula.

In order to achieve a better blocking effect, the minimum outer diameter of the fistula blocking section 21 is greater than the maximum inner diameter of the fistula. In order to prevent secondary injury to the patient due to the fistula being excessively expanded by the fistula blocking section 21, the minimum outer diameter of the fistula blocking section 21 is slightly greater than the maximum inner diameter of the fistula.

In an embodiment, the outer diameter of the fistula blocking section 21 is from 4 mm to 30 mm. Optionally, the outer diameter of the fistula blocking section 21 may be 4 mm, 6 mm, 9 mm, 12 mm, 15 mm, 18 mm, 20 mm, 22 mm, 25 mm, or 30 mm. The operator can select the fistula blocking stent 100 with an appropriate size according to a size of the fistula.

The first fixing stent 1 and the second fixing stent 2 are woven by threads of the same or different materials. In an embodiment, both the first fixing stent 1 and the second fixing stent 2 are woven by metal wires. In an embodiment, the first fixing stent 1 is woven by metal wires, and the second fixing stent 2 is woven with non-metallic degradable threads. Specifically, the metal wire is made of nickel-titanium alloy or absorbable magnesium alloy with good biocompatibility, and the non-metallic degradable thread is made of PLA (Polylactic Acid) or PLG (Lactide-co-Glycolide).

Manufacturing processes of the first fixing stent 1 and the second fixing stent 2 is not limited to the weaving process, but can also adopt 3D printing process, engraving process, etc.

When the second fixing stent 2 is made of a degradable material, during the slow degradation process of the second fixing stent 2, the fistula is closed at the end of the second lumen with a clip or a nylon bandage. Stem cells or platelet-rich plasma (PRP) can be injected to promote the healing of the fistula tissue. When the fistula is completely closed and healed, and the injection of methylene blue is confirmed to be correct, the first fixing stent 1 is pulled out of the first lumen, and the treatment of the fistula is completed.

An outer surface of the metal wire is provided with a corrosion-resistant coating or an antibacterial coating. The corrosion-resistant coating is composed of polysilicone, and the antibacterial coating is composed of one or more of organic silicon cationic steroid, minocycline, rifampicin, gentamicin, vancomycin and hydrophobic material.

In an optional embodiment, the fistula blocking stent 100 further includes a traction wire 4 circumferentially provided at the second end 220. The second end 220 can be contracted by pulling the traction wire 4, a volume of the second end 220 in the contracted state is small, so that the second end 220 can be smoothly pulled into the second lumen from the first lumen through the fistula, thereby reducing the delivery difficulty and improving the delivery efficiency. At the same time, a friction between the second fixing stent 2 and the fistula is reduced during the delivery process, which reduces the probability of secondary injury to the patient.

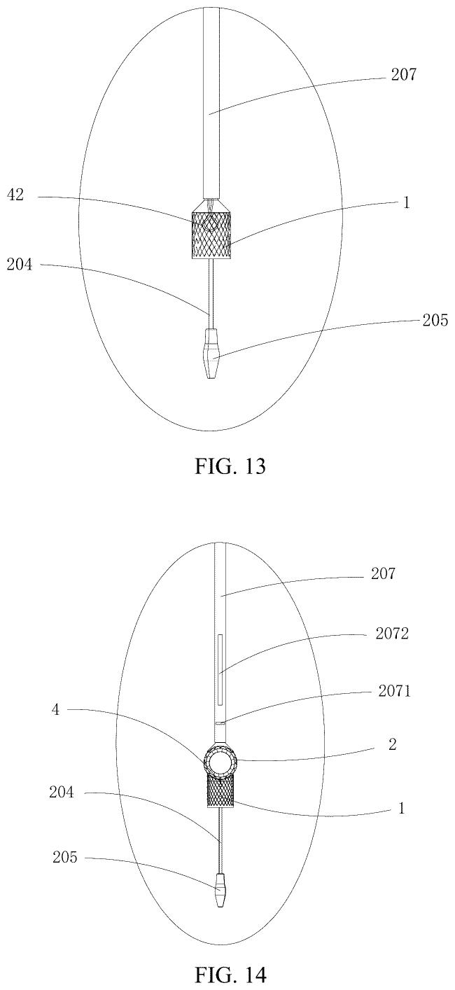

Referring to FIG. 1, FIG. 3, FIG. 10 and FIG. 13, the second end 220 can be contracted by pulling the traction wire 4, and the volume of the second end 220 in the contracted state is small, so that the second end 220 can be smoothly pulled into the second lumen from the first lumen through the fistula, thereby reducing the delivery difficulty and improving the delivery efficiency. At the same time, the friction between the second fixing stent 2 and the fistula is reduced during the delivery process, which reduces the probability of causing secondary injury to the patient. The traction wire 4 is released, and the second end 220 is expanded to position the second fixing stent 2.

The traction wire 4 is circumferentially provided at the second end 220. When the second end 220 is in the contracted state, a free section 42 of the traction wire 4 extends out of the second end 220 and is placed toward the distal end of the first fixing stent 1 for the operator to grasp and apply force to contract and move the second fixing stent 2.

Prior to placing the fistula blocking stent 100 into the delivering device 200, a force is applied to the free section 42 of the traction wire 4 to contract the second end 220 of the second fixing stent 2, so that the second fixing stent 2 is conveniently pulled into the second lumen through the fistula during the release of the fistula blocking stent 100. After the fistula blocking stent 100 is placed in the target area, the delivering device 200 is operated to slowly release the fistula blocking stent 100. When the operator observes that the free section 42 of the traction wire 4 is exposed from the delivering device 200, the operator pulls the free section 42 to apply an external force to the second fixing stent 2 toward the second lumen, thereby gradually pulling the second fixing stent 2 into the second lumen through the fistula. After the fistula blocking stent 100 is completely released, the external force is removed, and the second fixing stent 2 expands under its own elastic force and memory tension to complete the blocking of the fistula and the fixing of the second fixing stent 2.

The material of the traction wire 4 may be the same as that of the second fixing stent 2, or may be a non-metallic material with good biocompatibility. Referring to FIGS. 1 and 3. The fistula blocking stent 100 further includes a retraction wire 5, and the retraction wire 5 circumferentially extends through the proximal end of the first fixing stent 1. The retraction wire 5 is used for the operator to grasp and apply force to pull the fistula blocking stent 100 out of the patient, and to contract the first fixing stent 1, so as to accommodate the contracted fistula blocking stent 100 into the delivering device 200.

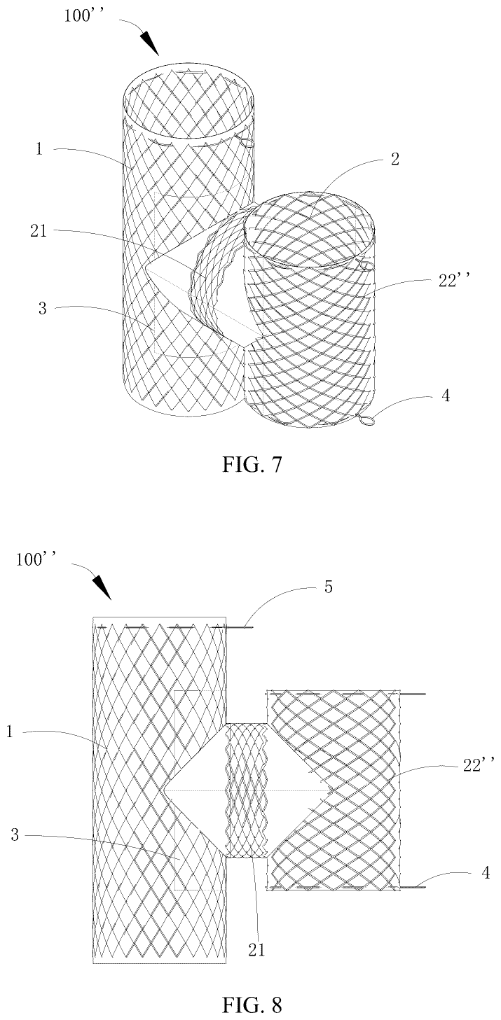

A shape of the flange 22 is not limited, and may be mushroom-shaped as shown in FIG. 1. In an embodiment, referring to FIG. 6, the flange 22′ of the fistula blocking stent 100′ is umbrella-shaped. In another embodiment, referring to FIG. 7 and FIG. 8, the flange 22″ of the fistula blocking stent 100″ is in a shape of a hollow tube, so that the fistula blocking stent 100″ is H-shaped as a whole. Compared with the above embodiments, the structures of the flanges 22′, 22″ are modified in the embodiments shown in FIG. 6, FIG. 7 and FIG. 8. For other same structures, the same reference numerals are used in the embodiments and will not be repeated.

Referring to FIG. 7 and FIG. 8, specifically, the flange 22″ of the fistula blocking stent 100″ is in a shape of the hollow tube, and a central axis of the flange 22″ is parallel to a central axis of the first fixing stent 1. In the first direction, a size of the flange 22″ is less than the size of the first fixing stent 1, and the flange 22″ is provided in the middle of the first fixing stent 1. The second end 220 includes a distal end and a proximal end of the flange 22″, and the traction wire 4 is provided at least one of the proximal end and the distal end of the flange 22″.

In other embodiments, the size of the flange 22″ in the first direction may be greater than or equal to the size of the first fixing stent 1.

Since an inner diameter of the human esophageal lumen generally ranges from 16 mm to 20 mm, and an inner diameter of the tracheal lumen ranges from 16 mm to 20 mm, when the inner diameter of the fistula is greater than 16 mm, a diameter of protrusions at the two ends of the fistula is 22 mm. When the fistula blocking stents 100, 100′ with the mushroom-shaped or umbrella-shaped flanges 22, 22′ is used for blocking the fistula, in order to achieve a better blocking effect, an outer diameter of the flange 22, 22′ needs to be greater than the inner diameter of the esophageal lumen or the inner diameter of the tracheal lumen, which may affect the normal swallowing of food or cause tracheal stenosis. When the flange 22 is umbrella-shaped, an edge of the flange 22′ may cut the tracheal wall and cause complications. Therefore, when the inner diameter of the fistula is greater than 16 mm, the fistula blocking stent 100″ as shown in FIG. 8 is used for blocking the fistula, and the fistula blocking stent 100″ is delivered from the esophageal lumen.

In an alternative embodiment, the first cover 31 and the first fixing stent 1, as well as the second cover 33 and the second fixing stent 2 are formed separately and then connected to each other. The shape and size of the first covering film 31 matches the shape and size of the first fixing stent 1, and the shape and size of the second covering film 33 matches the shape and size of the second fixing stent 2. After the first fixing stent 1, the second fixing stent 2, the first covering film 31, and the second covering film 33 are respectively manufactured, the first covering film 31 is attached to the inner side or the outer side of the first fixing stent 1, and the second covering film 33 is attached to the inner side or outer side of the second fixing stent 2. The covering film and the fixing stent can be connected by a hot melting process or the elasticity of the covering film and the fixing stent. When the covering film is connected to the fixing stent by the elasticity of the covering film and the fixing stent, when the covering film is arranged on the outer side of the fixing stent, the outer diameter of the fixing stent is greater than or equal to the inner diameter of the covering film, so that the fixing stent and the covering film can be closely attached. When the covering film is arranged on the inner side of the fixing stent, the inner diameter of the fixing stent is less than or equal to the outer diameter of the covering film, so that the fixing stent and the covering film can be closely attached.

In an alternative embodiment, the traction wire 4 may be omitted. In that case, the second end 220 of the second fixing stent 2 can be contracted in other ways, or the second end 220 of the second fixing stent 2 may not be contracted.

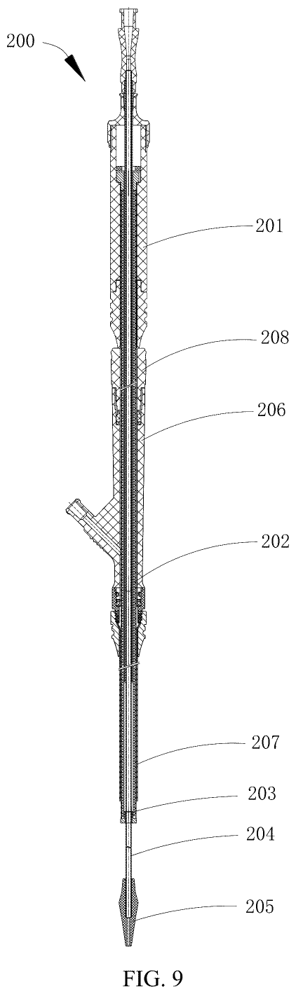

Referring to FIG. 9 to FIG. 14, according to some embodiments, a stent delivering system is provided, which includes the delivering device 200 and the fistula blocking stents 100, 100′ or 100″. The fistula blocking stents 100, 100′, or 100″ is delivered into the patient through the delivering device 200.

The delivering device 200 includes a first handle 201, a boosting tube 202 connected to the first handle 201, a middle tube 203 connected to the boosting tube 202, an inner tube 204 connected to the middle tube 203, a pushing head 205 connected to the inner tube 204, a second handle 206, an outer tube 207 connected to the second handle 206, and a safety lock 208 configured to lock and unlock a relative position between the second handle 206 and the boosting tube 202. The first handle 201 is closer to the operator than the second handle 206. The boosting tube 202 extends through the second handle 206 and is connected to the middle tube 203, the outer tube 207 is wrapped around the boosting tube 202, the middle tube 203 and the inner tube 204, and the fistula blocking stents 100, 100′, 100″ are contracted and delivered between the inner tube 204 and the outer tube 207. When the safety lock 208 is opened, the second handle 206 can be operated to move relative to the boosting tube 202, which can drive the outer tube 207 to move relative to the inner tube 204. After the outer tube 207 moves in place, the safety lock 208 is operated to lock the position of the second handle 206 relative to the boosting tube 202.

An outer diameter of the inner tube 204 is less than an outer diameter of the middle tube 203, so that an accommodation space is formed between an inner wall of the outer tube 207 and an outer wall of the inner tube 204, and the accommodation space is configured to accommodate the fistula blocking stents 100, 100′, 100″ in the contracted state.

The middle tube 203 includes a distal end connected to the inner tube 204. The pushing head 205 includes a proximal end connected to the inner tube 204. The outer diameter of the inner tube 204 is less than an outer diameter of the distal end of the middle tube 203 and an outer diameter of a proximal end of the pushing head 205. The outer wall of the inner tube 204, the inner wall of the outer tube 207, a distal end surface of the middle tube 203 and a proximal end surface of the pushing head 205 cooperatively form the accommodation space. Since the outer diameter of the inner tube 204 is less than the outer diameter of the middle tube 203 and the outer diameter of the proximal end of the pushing head 205, steps are formed at a connection between the inner tube 204 and the middle tube 203 and a connection between the inner tube 204 and the pushing head 205, so that a axial relative positional relationship between any one of the fistula blocking stents 100, 100′, 100″ in the contracted state and the inner tube 204 mounted in the accommodation space is fixed, thereby improving the delivery accuracy.

The safety lock 208 is released, and the second handle 206 is operated to move the outer tube 207 toward the distal end of the inner tube 204, so that the fistula blocking stent 100, 100′, or 100″ is gradually contracted into the accommodation space. After the fistula blocking stent 100, 100′, or 100″ is fully contracted into the accommodation space, the safety lock 208 is operated to lock the relative position of the second handle 206 and the boosting tube 202, and the first handle 201 is operated to deliver the fistula blocking stent 100, 100′ or 100″ into the patient. When the fistula blocking stent 100, 100′, or 100″ need to be released, the safety lock 208 is released, and the second handle 206 is operated to retract the outer tube 207, so that the fistula blocking stent 100, 100′, or 100″ is gradually released from the outer tube 207.

In order to improve the delivery accuracy of the fistula blocking stent 100, 100′, or 100″, a positioning mark 2071 is provided on the outer tube 207. When the positioning mark 2071 reaches a predetermined position, it means the fistula blocking stent 100, 100′, or 100″ is also placed in the target area. The target area refers to an area where the outer tube 207 can start to release the fistula blocking stent 100, 100′, or 100″.

Specifically, in an embodiment, the positioning mark 2071 is provided at an end of the outer tube 207 adjacent to the second handle 206. In this embodiment, before the fistula blocking stent 100, 100′, or 100″ is delivered into the first lumen, a position information of the fistula is obtained through an image scanning device or an endoscope, etc. According to the position information of the fistula and a size of the delivering device 200, an insertion depth of the delivering device 200 can be obtained, so as to obtain a relative position relationship between the outer tube 207 and the mouth or other organs of the patient when the delivering device 200 is placed in the target area. The positioning mark 2071 is marked at a corresponding position of the outer tube 207. When the positioning mark 2071 reaches the corresponding position of the patient, the fistula blocking stent 100, 100′, or 100″ is also placed in the target area, and the insertion of the delivering device 200 into the first lumen is stopped.

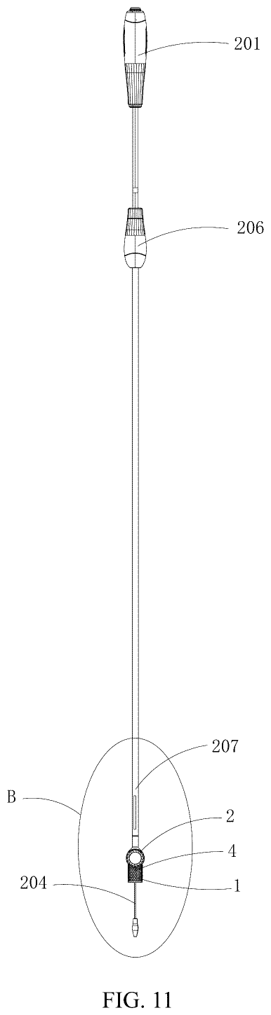

In an alternative embodiment, the positioning mark 2071 may be provided at an end of the outer tube 207 away from the second handle 206. Referring to FIGS. 11 and 13, in this embodiment, a first lumen endoscope is delivered into the first lumen prior to delivering the fistula blocking stent 100, 100′, or 100″ into the first lumen. The first lumen endoscope is in a position where it can capture the position information of the fistula. The positioning mark 2071 is provided in an area of the outer tube 207 corresponding to the first connection track of the fistula blocking stent 100, 100′, or 100″. When the first lumen endoscope captures the positioning mark 2071 and the positioning mark 2071 is located in the fistula in the first direction, it means the fistula blocking stent 100, 100′, or 100″ is placed in the target area, and the insertion of the delivering device 200 into the first lumen is stopped.

During the process of delivering the fistula blocking stent 100, 100′, or 100″ into the target area, the entire delivering device 200 may rotate by an undetermined angle, so that the free section 42 of the traction wire 4 in a circumferential direction may not be within an observation range of a second lumen endoscope, thereby preventing the operator from capturing the traction wire 4 in time and pulling the traction wire 4 into the second lumen. In this case, the first handle 201 needs to be rotated to enable the free section 42 of the traction wire 4 to be within the observation range of the second lumen endoscope. In addition, in order to facilitate a forceps to clamp the free section 42 after passing through the fistula, the free section 42 needs to face the fistula. However, the lighting in the first lumen is weak, and it is difficult for the second lumen endoscope to accurately identify the position of the free section 42 through the outer tube 207.

In order to solve the above problem, the outer tube 207 of the delivering device 200 is further provided with a positioning mark 2072, which is provided on an outer surface of the outer tube 207 and is a conspicuous line segment. The positioning mark 2072 is arranged corresponding to the free section 42 of the fistula blocking stent 100, 100′, or 100″ in the contracted state. After the fistula blocking stent 100, 100′, or 100″ is placed in the target area, the first handle 201 is rotated, so that the positioning mark 2072 is within the observation range of the second lumen endoscope, which is convenient for the operator to pull the traction wire 4 into the second lumen accurately and timely.

In alternative embodiments, the positioning mark 2072 is detachably attached to the outer tube 207.

In an embodiment, the fistula blocking stent 100 is delivered from the tracheal lumen. After the delivery is completed, the first fixing stent 1 is placed in the trachea, and the second fixing stent 2 extends through the fistula and is partially placed in the esophagus. Compared with the esophageal lumen, there is no liquid or solid passing through the tracheal lumen, and the inner diameter of the tracheal lumen fluctuates less. Therefore, the positioning stability of the fistula blocking stent 100, 100′, or 100″ can be ensured by placing the first fixing stent 1 in the tracheal lumen and abutting against the inner wall of the tracheal lumen.

In the embodiment in which the traction wire 4 is not provided, the positioning mark 2072 is arranged corresponding to the second fixing stent 2, the forceps clamps the second fixing stent 2 after passing through the fistula. The first handle 201 is rotated after the fistula blocking stent 100, 100′, or 100″ is placed in the target area, so that the positioning mark 2072 is in the observation range of the second endoscopy, which is convenient for the operator to pull the second fixing stent 2 into the second lumen accurately and timely.

According to an embodiment, a method of delivering the fistula blocking stent 100 using the aforementioned stent delivering system is provided. In one embodiment, the fistula blocking stent 100 or 100′ may be selected, the first lumen is the tracheal lumen, the second lumen is the esophageal lumen, and the fistula blocking stent 100, 100′ is delivered from the tracheal lumen. The method includes the following steps.

S11: The fistula blocking stent 100, or 100′ is contracted into the accommodation space of the delivering device 200.

Specifically, the first fixing stent 1 of the fistula blocking stent 100 or 100′ is sleeved on the inner tube 204 of the delivering device 200, the operator applies a force to the retraction wire 5 and the traction wire 4 to contract the proximal end of the first fixing stent 1 and the second end 220 of the second fixing stent 2. The safety lock 208 is released, and the operator operates the second handle 206 of the delivering device 200, so that the outer tube 207 of the delivering device 200 moves toward the distal end of the delivering device 200 and receives the first fixing stent 1 is gradually received into the outer tube 207. The second handle 206 of the delivering device 200 is continuously operated, the outer tube 207 continue to move toward the distal end of the delivering device 200 until the fistula blocking stent 100 or 100′ is completely received into the accommodation space between the outer tube 207 and the inner tube 204, then the safety lock 208 is locked.

S12: The delivering device 200 is inserted into the first lumen.

Specifically, a cannula is inserted into the first lumen, the first lumen endoscope enters the first lumen through the cannula, and passes the fistula. After a guiding wire is indwelled, the first lumen endoscope is removed, and the delivering device 200 enters the first lumen from the cannula through the guiding wire.

S13: The positioning mark 2072 of the delivering device 200 is positioned.

Specifically, the second lumen endoscope is inserted to reach the fistula from the second lumen. Whether the fistula blocking stent 100 or 100′ in the first lumen is placed in position can be observed through the second lumen endoscope via the fistula. The delivering device 200 is rotated until the positioning mark 2072 can be observed through the second lumen endoscope and the positioning mark 2072 is orientated towards the fistula.

S14: The fistula blocking stent 100 or 100′ is partially released, and the free section 42 of the traction wire 4 is pulled into the second lumen.

Specifically, the safety lock 208 is released, the second handle 206 is operated to retract the outer tube 207 to gradually release the fistula blocking stent 100 or 100′. When the free section 42 of the traction wire 4 is observed from the second lumen endoscope, releasing of the fistula blocking stent 100 or 100′ is paused, the forceps extends through a forceps channel of the second lumen endoscope and passes the fistula and enters the first lumen to grasp the free section 42 of the traction wire 4, and the free section 42 of the traction wire 4 is pulled into the second lumen through the fistula.

S15: The fistula blocking stent 100 or 100′ is completely released.

Specifically, the outer tube 207 is continuously retracted to gradually release the fistula blocking stent 100 or 100′, and at the same time, the forceps continue to apply a force to the free section 42 toward the second lumen, so that the second fixing stent 2 is gradually pulled into the second lumen through the fistula by the forceps during the gradual release of the fistula blocking stent 100 or 100′. The outer tube 207 is continuously retracted until the proximal end of the first fixing stent 1 is released from the outer tube 207.

S16: The positioning of the fistula blocking stent 100 or 100′ is completed.

Specifically, the forceps releases the free section 42 of the traction wire, so that the second end 220 of the second fixing stent 2 is released to be expanded, and the fistula blocking stent 100, 100′ is recovered to the initial state. The flange 22 is located in the second lumen and abuts against the inner wall of the second lumen, so as to prevent the second fixing stent 2 from moving from the second lumen to the first lumen. The fistula blocking section 21 abuts against the inner wall of the fistula, so as to block the fistula and support of the fistula. The first fixing stent 1 elastically abuts against the inner wall of the first lumen elastically to achieve the fixation of the fistula blocking stent 100 or 100′.

S17: The delivering device 200 and the guiding wire is retracted from the first lumen. S18: Whether the fistula blocking operation is successful is determined.

Specifically, staining solution is injected to a position of the second lumen adjacent to the fistula through the second lumen endoscope. The first lumen endoscope reaches the fistula blocking stent 100 or 100′ from the first lumen, and it is determined that whether the staining solution leaks into the first lumen from the second lumen is observed through the first lumen endoscope, if not, the fistula blocking operation is determined to be successful.

When the inner diameter of the fistula is greater than 16 mm, the fistula blocking stent 100″ as shown in FIG. 8 is used for blocking the fistula, and the fistula blocking stent 100″ is delivered from the esophageal lumen. In addition, after esophagectomy, a stomach needs to be moved upward to suture both ends of the esophagus. In this case, the anastomotic fistula formed is relatively narrow, so that it is appropriate to place the first fixing stent 1 in the esophageal lumen and to place the second fixing stent 2 in the tracheal lumen to block the fistula. In an embodiment, the stent 100, 100′ or 100″ is delivered from the esophageal lumen, after which the first fixing stent 1 is placed in the esophagus, and the second fixing stent 2 is placed in the tracheal lumen after passing through the fistula.

When the fistula blocking stent 100, 100′, 100″ is delivered from the esophageal lumen, the first lumen is the esophageal lumen, and the second lumen is the tracheal lumen. The method includes the following steps.

S21: The fistula blocking stent 100, 100′ or 100″ is contracted into the accommodation space of the delivering device 200.

Specifically, the first fixing stent 1 of the fistula blocking stent 100, 100′ or 100″ is sleeved on the inner tube 204 of the delivering device 200, the operator applies a force to the retraction wire 5 and the traction wire 4 to contract the proximal end of the first fixing stent 1 and the second end 220 of the second fixing stent 2. The safety lock 208 is released, and the operator operates the second handle 206 of the delivering device 200, so that the outer tube 207 of the delivering device 200 moves toward the distal end of the delivering device 200 and receives the first fixing stent 1 is gradually received into the outer tube 207. The second handle 206 of the delivering device 200 is continuously operated, the outer tube 207 continue to move toward the distal end of the delivering device 200 until the fistula blocking stent 100, 100′ or 100″ is completely received into the accommodation space between the outer tube 207 and the inner tube 204, then the safety lock 208 is locked.

S22: The delivering device 200 is inserted into the first lumen.

Specifically, the first lumen endoscope is inserted from the first lumen, and passes the fistula. After a guiding wire is indwelled, the first lumen endoscope is removed, and the delivering device 200 enters the first lumen through the guiding wire.

S23: The positioning mark 2072 of the delivering device 200 is positioned.

Specifically, a cannula is inserted into the second lumen, and the second lumen endoscope is inserted into the second lumen from the cannula and enters the fistula. Whether the fistula blocking stent 100, 100′ or 100″ in the first lumen is placed in position can be observed through the second lumen endoscope via the fistula. The delivering device 200 is rotated until the positioning mark 2072 can be observed through the second lumen endoscope and the positioning mark 2072 is orientated towards the fistula.

S24: The fistula blocking stent 100, 100′ or 100″ is partially released, and the free section 42 of the traction wire 4 is pulled into the second lumen.

Specifically, the safety lock 208 is released, the second handle 206 is operated to retract the outer tube 207 to gradually release the fistula blocking stent 100, 100′ or 100″. When the free section 42 of the traction wire 4 is observed from the second lumen endoscope, releasing of the fistula blocking stent 100, 100′ or 100″ is paused, the forceps extends through a forceps channel of the second lumen endoscope and passes the fistula and enters the first lumen to grasp the free section 42 of the traction wire 4, and the free section 42 of the traction wire 4 is pulled into the second lumen through the fistula.

S25: The fistula blocking stent 100, 100′ or 100″ is completely released.

Specifically, the outer tube 207 is continuously retracted to gradually release the fistula blocking stent 100, 100′ or 100″, and at the same time, the forceps continue to apply a force to the free section 42 toward the second lumen, so that during the gradual release of the fistula blocking stent 100, 100′ or 100″, the second fixing stent 2 is gradually pulled into the second lumen through the fistula by the forceps. The outer tube 207 is continuously retracted until the proximal end of the first fixing stent 1 is released from the outer tube 207.

S26: The positioning of the fistula blocking stent 100, 100′ or 100″ is completed.

Specifically, the forceps releases the free section 42 of the traction wire, so that the second end 220 of the second fixing stent 2 is released to be expanded, and the fistula blocking stent 100, 100′ or 100″ is recovered to the initial state. The flange 22 is located in the second lumen and abuts against the inner wall of the second lumen, so as to prevent the second fixing stent 2 from moving from the second lumen to the first lumen. The fistula blocking section 21 abuts against the inner wall of the fistula, so as to block the fistula and support of the fistula. The first fixing stent 1 elastically abuts against the inner wall of the first lumen elastically to achieve the fixation of the fistula blocking stent 100, 100′ or 100″.

S27: The delivering device 200 and the guiding wire is removed from the first lumen.

S28: Whether the fistula blocking operation is successful is determined.

Specifically, the first lumen endoscope is inserted into the first lumen. The staining solution is injected to a position of the first lumen adjacent to the fistula through the first lumen endoscope, and it is determined that whether the staining solution leaks into the second lumen from the first lumen is observed through the second lumen endoscope, if not, the fistula blocking operation is determined to be successful.

In an embodiment, the staining solution is methylene blue.

In alternative embodiments, when delivery of the fistula blocking stent 100, 100′, 100″ is completed, the fistula blocking operation is determined to be successful, and the step of determining whether the fistula blocking operation is successful can be omitted.

In alternative embodiments, the stent delivering system may not be provided with the positioning mark 2072, and correspondingly, the method does not include the step of positioning the positioning mark 2072.

In an embodiment, the guiding wire is a zebra guiding wire having a diameter of 0.89 mm. The outer diameter of the outer tube 207 is less than or equal to 6.7 mm, and an inner diameter of the cannula is 7.5 mm. These dimensions are for illustrative purposes and are not limited hereto.

In the embodiment without the traction wire 4, the second end 220 of the second fixing stent 2 can be contracted in other ways. Alternatively, the delivery of the fistula blocking stent 100, 100′ or 100″ is performed with the second end 220 is in an initial state. During the delivery process, the forceps may directly apply a force to the second fixing stent 2, or apply a force to the second fixing stent 2 through other components, so as to pull the second fixing stent 2 into the second lumen.

In the description of the present disclosure, it should be understood that the terms “center”, “longitudinal”, “transverse”, “length”, “width”, “thickness”, “upper”, “lower”, “front”, “rear”, “left”, “right”, “vertical”, “horizontal”, “top”, “bottom”, “inner”, “outer”, “clockwise”, “counterclockwise”, “axial”, “radial”, “circumferential direction” are based on the azimuth or position relationship shown in the attached drawings, which are only for the convenience of describing the present disclosure and simplifying the description, rather than indicating or implying that the device or element must have a specific azimuth, be constructed and operated in a specific azimuth, so such terms cannot be understood as a limitation of the present disclosure.

In addition, the terms “first” and “second” are only used for descriptive purposes and cannot be understood as indicating or implying relative importance or implicitly indicating the number of indicated technical features. Thus, the features defined with “first” and “second” may explicitly or implicitly include at least one of the features. In the description of the present disclosure, “a plurality of” means at least two, such as two, three, etc., unless otherwise expressly and specifically defined.

In the present disclosure, unless otherwise expressly specified and limited, the terms “mount”, “connect”, “contact”, “fix” and other terms should be understood in a broad sense, for example, they can be fixed connections, detachable connections, or integrated. They can be mechanical connection or electrical connection. They can be directly connected or indirectly connected through an intermediate medium. They can be the connection within two elements or the interaction relationship between two elements, unless otherwise expressly limited. For those skilled in the art, the specific meaning of the above terms in the present disclosure can be understood according to the specific situation.

In the present disclosure, unless otherwise expressly specified and limited, the first feature “above” or “below” the second feature may be in direct contact with the first and second features, or the first and second features may be in indirect contact through an intermediate medium. Moreover, the first feature is “above” the second feature, but the first feature is directly above or diagonally above the second feature, or it only means that the horizontal height of the first feature is higher than the second feature. The first feature is “below” of the second feature, which can mean that the first feature is directly below or obliquely below the second feature, or simply that the horizontal height of the first feature is less than that of the second feature.

It should be noted that when an element is called “fixed to” or “provided on” another element, it can be directly on another element or there can be a centered element. When an element is considered to be “connected” to another element, it can be directly connected to another element or there may be intermediate elements at the same time. The terms “vertical”, “horizontal”, “up”, “down”, “left”, “right” and similar expressions used herein are for the purpose of illustration only and do not represent the only embodiment.

The above-mentioned embodiments do not constitute a limitation on the protection scope of the technical solution. Any modifications, equivalent redeliverys and improvements made within the spirit and principles of the above-mentioned embodiments shall be included within the protection scope of this technical solution.

The foregoing descriptions are merely specific embodiments of the present disclosure, but are not intended to limit the protection scope of the present disclosure. Any variation or redelivery readily figured out by a person skilled in the art within the technical scope disclosed in the present disclosure shall all fall within the protection scope of the present disclosure.

What is claimed is:

1. A fistula blocking stent configured to block a fistula formed between a first lumen and a second lumen, the fistula blocking stent comprising:
a first fixing stent in a shape of a hollow tube, the first fixing stent having a proximal end, a distal end, and a tube body extending axially between the proximal end and the distal end, the first fixing stent being provided in the first lumen to support an inner wall of the first lumen;
a second fixing stent provided in the fistula and the second lumen, the second fixing stent having a first end and a second end opposite to the first end, the first end being connected to the first fixing stent; and
a barrier film provided on at least one of the first fixing stent and the second fixing stent and configured to block the fistula.

2. The fistula blocking stent according to claim 1, further comprising a traction wire provided at the second end, wherein the second end is contracted by pulling the traction wire.

3. The fistula blocking stent according to claim 2, wherein when the traction wire is pulled, the second end is contracted to enable the second fixing stent to enter the second lumen through the fistula, when the traction wire is released, the second end is expanded to position the second fixing stent.

4. The fistula blocking stent according to claim 2, wherein the traction wire is circumferentially provided at the second end, when the second end is in a contracted state, a free section of the traction wire extends out of the second end and is placed toward the distal end of the first fixing stent.

5. The fistula blocking stent according to claim 1, wherein the first fixing stent and the second fixing stent are connected by a suture.

6. The fistula blocking stent according to claim 1, wherein the barrier film comprises a first covering film covering the first fixing stent, the second fixing stent is connected to the first fixing stent to form a first connection track, and the first covering film covers the first connection track.

7. The fistula blocking stent according to claim 1, wherein the barrier film comprises a first barrier film connected to at least one of the first and second fixing stent, the second fixing stent is connected to the first fixing stent to form a first connection track, and the first barrier film covers the first connection track.

8. The fistula blocking stent according to claim 7, wherein the first barrier film is connected to the first fixing stent to form a second connection track, an area enclosed by the second connection track is greater than an area enclosed by the first connection track, and the first connection track is located within the second connection track.

9. The fistula blocking stent according to claim 7, wherein the first barrier film is an acid-resistant film.

10. The fistula blocking stent according to claim 7, wherein the barrier film further comprises a second covering film covering the second fixing stent and a second barrier film provided in the second fixing stent, and the second barrier film is provided in a space surrounded by the second covering film to block the fistula.

11. The fistula blocking stent according to claim 10, wherein the first end is provided with a fistula blocking section, the second end is provided with a flange, a diameter of the flange is greater than a diameter of the fistula blocking section, and the second covering film covers the fistula blocking section.

12. The fistula blocking stent according to claim 10, wherein the first end is provided with a fistula blocking section, the second end is provided with a flange, the flange is in a shape of a hollow tube, and a central axis of the flange is parallel to a central axis of the first fixing stent.

13. The fistula blocking stent according to claim 1, wherein the first fixing stent is woven by metal wires, and the second fixing stent is woven by degradable wire or metal wires.

14. A stent delivering system, comprising:
a delivering device comprising:
a first handle, a boosting tube connected to the first handle, a middle tube connected to the boosting tube, an inner tube connected to the middle tube, a pushing head connected to the inner tube;
a second handle, an outer tube connected to the second handle, and a safety lock configured to lock or unlock a relative position between the second handle and the boosting tube, wherein the first handle is closer to an operator than the second handle, the booster tube extending through the second handle and being connected to the middle tube, a diameter of the inner tube being less than a diameter of a distal end of the middle tube and a diameter of a proximal end of the pushing head, and the inner tube, the distal end of the middle tube, the proximal end of the pushing head, and the outer tube cooperatively form an accommodation space; and
the fistula blocking stent according to claim 1 provided in the accommodation space, wherein when the safety lock is released and the second handle is operated to retract the outer tube, the fistula blocking stent is released from the accommodation space.

15. The stent delivering system according to claim 14, wherein the fistula blocking stent further comprises a traction wire circumferentially provided at the second end, when the second end is in a contracted state, a free section of the traction wire extends out of the second end and is placed toward the distal end of the first fixing stent, an outer surface of the outer tube is provided with a positioning mark corresponding to the free section.

16. A method of delivering a fistula blocking stent using the stent delivering system of claim 15, comprising:
contracting the fistula blocking stent into the accommodation space, and tightening locking the safety lock;
inserting a cannula into the first lumen, enabling a first lumen endoscope to enter the first lumen through the cannula and pass a fistula, indwelling a guiding wire, removing the first lumen endoscope, and enabling the delivering device to enter the first lumen from the cannula through the guiding wire;
inserting a second lumen endoscope to reach the fistula from the second lumen, unlocking the safety lock, operating the second handle to retract the outer tube to gradually release the fistula blocking stent, and when the traction wire is observed from the second lumen endoscope, pausing releasing of the fistula blocking stent, grabbing, by a forceps extending through a forceps channel of the second lumen endoscope and passing the fistula and entering the first lumen, the traction wire, and pulling the traction wire into the second lumen through the fistula;
continuing to retract the outer tube to gradually release the fistula blocking stent, enabling the forceps to continuously apply a force to the traction wire toward the second lumen, so that the second fixing stent is gradually pulled into the second lumen through the fistula by the forceps during the gradual release of the fistula blocking stent, continuing to retract the outer tube until the proximal end of the first fixing stent is released from the outer tube;
releasing, by the forceps, the traction wire, so that the second end of the second fixing stent is released to be expanded, wherein the second end is located in the second lumen and abuts against an inner wall of the second lumen, so as to prevent the second fixing stent from moving from the second lumen to the first lumen; and
removing the delivering device and the guiding wire from the first lumen.

17. The method according to claim 16, wherein when the second end is in a contracted state, a free section of the traction wire extends out of the second end is placed toward the distal end of the first fixing stent, an outer surface of the outer tube is provided with a positioning mark corresponding to the free section, wherein prior to operating the second handle to retract the outer tube to gradually release the fistula blocking stent, the method further comprises:
rotating the delivering device until the positioning mark is observed through the second lumen endoscope, and the positioning mark is orientated towards the fistula.

18. The method according to claim 16, further comprising:
injecting staining solution to a position of the second lumen adjacent to the fistula through the second lumen endoscope, enabling the first lumen endoscope to reach the fistula blocking stent from the first lumen, and determining whether the staining solution leaks into the first lumen from the second lumen through the first lumen endoscope.

19. A method of delivering a fistula blocking stent using the stent delivering system of claim 15, comprising:
contracting the fistula blocking stent into the accommodation space, and locking the safety lock;
inserting a first lumen endoscope from the first lumen, enabling the first lumen endoscope to pass a fistula, indwelling a guiding wire, removing the first lumen endoscope, and enabling the delivering device to enter the first lumen through the guiding wire;
inserting a cannula into the second lumen, inserting a second lumen endoscope into the second lumen from the cannula and enabling the second lumen endoscope to reach the fistula, unlocking the safety lock, operating the second handle to retract the outer tube to gradually release the fistula blocking stent, and when the traction wire is observed from the second lumen endoscope, pausing releasing of the fistula blocking stent, grabbing by a forceps extending through a forceps channel of the second lumen endoscope and passing the fistula and entering the first lumen, the traction wire, and pulling the traction wire into the second lumen through the fistula;
continuing to retract the outer tube to gradually release the fistula blocking stent, enabling the forceps to continuously apply a force to the traction wire toward the second lumen, so that the second fixing stent is gradually pulled into the second lumen through the fistula by the forceps during the gradual release of the fistula blocking stent, continuing to retract the outer tube until the proximal end of the first fixing stent is released from the outer tube;
releasing, by the forceps, the traction wire, so that the second end of the second fixing stent is released to be expanded, wherein the second end is located in the second lumen and abuts against an inner wall of the second lumen, so as to prevent the second fixing stent from moving from the second lumen to the first lumen; and
removing the delivering device and the guiding wire from the first lumen.

20. The method according to claim 19, wherein when the second end is in a contracted state, a free section of the traction wire extends out of the second end is placed toward the distal end of the first fixing stent, an outer surface of the outer tube is provided with a positioning mark corresponding to the free section, wherein prior to operating the second handle to retract the outer tube to gradually release the fistula blocking stent, the method further comprises:
rotating the delivering device until the positioning mark is observed through the second lumen endoscope, and the positioning mark is orientated towards the fistula.
